(19) United States
(12) Patent Application Publication
(10) Pub. No.: US 2019/0037938 A1
(43) Pub. Date: Feb. 7, 2019

(54) FUNCTIONAL GLOVE FOR HOLDING ULTRASONIC PROBE

(71) Applicant: AJOU UNIVERSITY INDUSTRY-ACADEMIC CORPORATION FOUNDATION, Suwon-si, Gyeonggi-do (KR)

(72) Inventor: Ji Man HONG, Yongin-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 16/077,047

(22) PCT Filed: Jun. 15, 2016
(86) PCT No.: PCT/KR2016/006344

§ 371 Date: Aug. 10, 2018

(30) Foreign Application Priority Data
Feb. 12, 2016 (KR) 10-2016-0016282

(51) Int. Cl. A41D 13/08; A41D 19/015; A61B 8/00

(52) U.S. Cl. CPC A41D 13/081; A61B 8/12; A61B 8/4472; A41D 19/015

(57) ABSTRACT

A functional glove for holding an ultrasonic probe includes: a body for encompassing the thumb and at least a part of the back of the hand of an inspector operating the ultrasonic probe; a probe fixing part coupled to the body so as to encompass and fix the fingers of the inspector and the ultrasonic probe; and a wrist protection part connected to the body so as to encompass and protect the wrist of the inspector.

### TECHNICAL FIELD

The present invention relates to a functional glove for holding an ultrasonic probe, and more particularly, to a functional glove for holding an ultrasonic probe which assists an inspector in easily holding the ultrasonic probe and with which the wrist of the inspector can be protected.

### BACKGROUND ART

In general, an ultrasonic imaging diagnosis device is medical imaging equipment that irradiates ultrasonic waves onto diagnostic areas of a subject and generates an image inside the diagnostic areas using a reflection signal of the ultrasonic waves.

The ultrasonic imaging diagnosis device can output an image in a real time compared to other imaging diagnosis devices such as X-ray, computerized tomography (CT), magnetic resonance imaging (MRI), and the like, and because the ultrasonic imaging diagnosis device does not any radiation exposure such as the X-ray, the ultrasonic imaging diagnosis device is widely used in diagnosis of the brain, the heart, the abdomen, the urinary system, and ob-syn.

The ultrasonic imaging diagnosis device includes an ultrasonic probe for transmitting ultrasonic signals into the human body and receiving reflected ultrasonic echo signals so as to attain an ultrasonic image of the human body. The ultrasonic probe is manufactured by arranging several tens of ultrasonic vibrators and putting the ultrasonic vibrators into a plastic case according to objects to be observed, and there are different shapes and sizes of the ultrasonic probe according to inspection areas and purposes.

The ultrasonic probe can be hold in various ways according to the shape and use purpose thereof. For example, the ultrasonic probe can be hold using the inspector's thumb, index finger and middle finger like an inspector holds a pen, and in some cases, the ultrasonic probe can also be hold to be encompassed like the inspector clenches his/her fist.

When the ultrasonic probe is hold as described above, strength is exerted in the fingers or the wrist of the inspector. When ultrasonic diagnosis is carried out for a long time with this posture, excessive strength is exerted in the fingers and the wrist of the inspector, and in severe cases, a wrist-joint injury of the inspector can occur.

Due to this problem, devices for assisting holding of the inspector while holding the ultrasonic probe have been developed. Korean Patent Registration No. 10-0969538 is disclosed as related arts thereof. This disclosure provides an ultrasonic probe including a body having an auxiliary holding part with a plurality of holes into which a user's fingers can be inserted.

However, in the ultrasonic probe according to the related art described above, the auxiliary holding part with a predetermined shape is formed in the body of the ultrasonic probe so that it is inconvenient to hold the ultrasonic probe with a different posture, and the ultrasonic probe according to the related art cannot be applied to an existing probe.

Furthermore, although, in the ultrasonic probe, excessive strength exerted in the fingers can be relieved during usage, a problem of exerting excessive strength in the wrist of the inspector has not been solved.

### DETAILED DESCRIPTION OF THE INVENTION

### Technical Problem

The present invention provides a functional glove for holding an ultrasonic probe, with which excessive strength can be prevented from being exerted in the hand and the wrist of an inspector and which can be utilized in various ultrasonic diagnosis devices.

### Technical Solution

According to an aspect of the present invention, there is provided a functional glove for holding an ultrasonic probe, including: a body for encompassing the thumb and at least a part of the back of the hand of an inspector operating the ultrasonic probe; a probe fixing part coupled to the body so as to encompass and fix the fingers of the inspector and the ultrasonic probe; and a wrist protection part connected to the body so as to encompass and protect the wrist of the inspector

The functional glove for holding an ultrasonic probe may further include a remote controller that is installed on the wrist protection part and is capable of remotely operating a main body of an ultrasonic imaging diagnosis device through short wireless communication.

The functional glove for holding an ultrasonic probe may further include sanitary gloves disposed inside the body so as to encompass the fingers and the back of the hand of the inspector and fixedly coupled to the body. The wrist protection part and neck parts of the sanitary gloves may be turned inside out in a direction of the fingers of the inspector.

The probe fixing part may include: a fixing band having one end fixed to the body so as to encompass and fix the fingers of the inspector and the ultrasonic probe; and an adhesive unit installed at both ends of the fixing band and configured to adhere or detach both ends of the fixing band to or from each other. The fixing band may have one end fixed to a part of the body corresponding to the thumb of the inspector and may have elasticity. The adhesive unit may include a Velcro or snap button with which both ends of the fixing band are detachably coupled to each other.

The wrist protection part may include: a protection band having one end connected to the body so as to encompass and protect the wrist of the inspector; and a detachment unit installed at both ends of the protection band and configured to adhere or detach both ends of the protection band to or from each other. The protection band may extend from the body as one body with the body and may have elasticity. The detachment unit may include a Velcro or snap button with which both ends of the protection band are detachably coupled to each other.

### Advantageous Effects

In a functional glove for holding an ultrasonic probe according to the present invention, the following effects can be obtained.

First, the fingers of an inspector and the ultrasonic probe are fixed while the ultrasonic probe is hold such that the ultrasonic probe can be hold without exerting excessive strength in the fingers of the inspector and thus work fatigue of the inspector can be reduced.

Second, the wrist of the inspector is pressed and fixed by a wrist protection part while the ultrasonic probe is hold, such that a wrist injury caused by a long diagnosis time can be prevented.

Third, the functional glove for holding an ultrasonic probe according to the present invention can also be applied to an existing ultrasonic probe such that the functional glove for holding an ultrasonic probe according to the present invention can be worn regardless of a holding method of the inspector and thus high utilization of various ultrasonic diagnosis devices can be achieved.

Fourth, disposable sanitary gloves are provided such that infection, which can occur between the inspector and a subject during diagnosis, can be prevented.

### DESCRIPTION OF THE DRAWINGS

FIG. 1 is a view of a wearing state of a functional glove for holding an ultrasonic probe according to the present invention;

FIG. 2 is a perspective view of a functional glove for holding an ultrasonic probe according to an embodiment of the present invention;

FIG. 3 is a perspective view of a wearing method of the functional glove for holding an ultrasonic probe illustrated in FIG. 2;

FIG. 4 is a perspective view of a functional glove for holding an ultrasonic probe according to another embodiment of the present invention;

FIG. 5 is a perspective view of a wearing method of the functional glove for holding an ultrasonic probe illustrated in FIG. 4; and

FIGS. 6 and 7 are views for illustrating using states of each of methods of holding an ultrasonic probe in a state in which the functional glove for holding an ultrasonic probe illustrated in FIG. 1 is worn.

### BEST MODE

Hereinafter, example embodiments of the present invention will be described in detail with reference to the accompanying drawings. Prior to this, the terms or words used in the present specification and the claims should not be interpreted to be limited in a general or dictionary sense and should be interpreted in a sense and a concept that comply with the technical spirit of the invention based on a principle that the inventor can properly define the concept of the terms so as to describe his/her own invention in a best manner.

Thus, configurations in the embodiments of the present specification and the drawings are just most example embodiments of the present invention and do not represent all of technical spirits of the invention. Thus, at a time for filing the present application, it should be understood that there may be equivalent modified examples that can replace them.

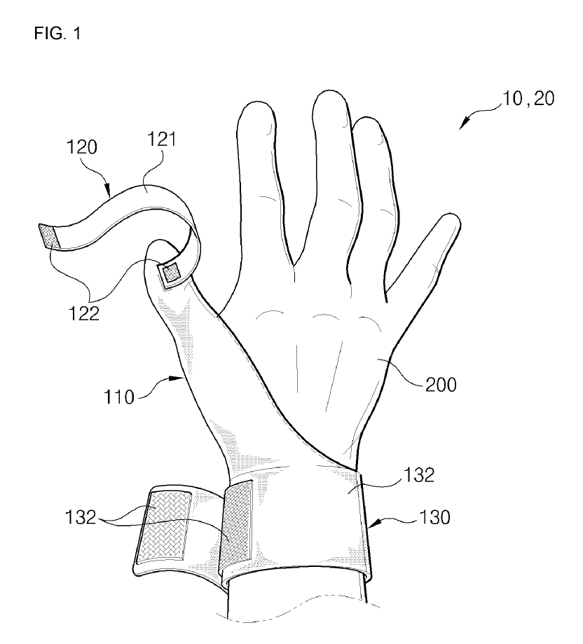

Hereinafter, embodiments of the present invention will be described below in detail with reference to the accompanying drawings. FIG. 1 is a view of a wearing state of a functional glove for holding an ultrasonic probe according to the present invention. Here, the ultrasonic probe that is equipment provided in an ultrasonic imaging diagnosis device is connected to a main body of the ultrasonic imaging diagnosis device. The main body forms an image using signals transmitted from the ultrasonic probe and outputs the image.

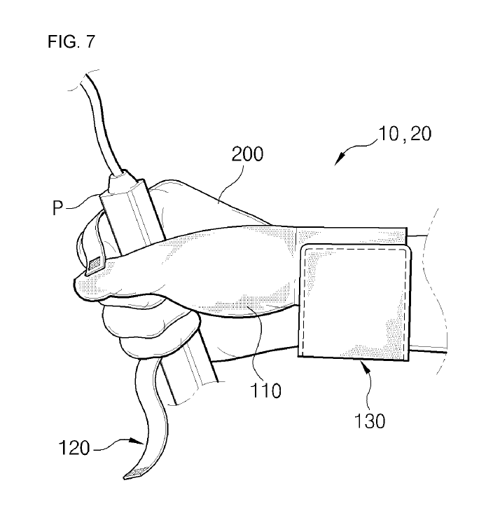

Referring to the drawings, a functional glove 10 or 20 for holding an ultrasonic probe according to the present invention is used to assist an inspector in easily holding an ultrasonic probe P (see FIGS. 6 and 7). To this end, the functional glove 10 or 20 includes a body 110, a probe fixing part 120, and a wrist protection part 130.

In the present invention described above, the functional glove 10 or 20 for holding the ultrasonic probe according to the present invention may further include disposable sanitary gloves 200. The sanitary gloves 200 are not greatly different from general gloves used for a medical purpose and thus, a detailed description thereof will be omitted.

Hereinafter, different embodiments will be described depending on whether the sanitary gloves 200 are fixedly coupled to an inside of the body 110.

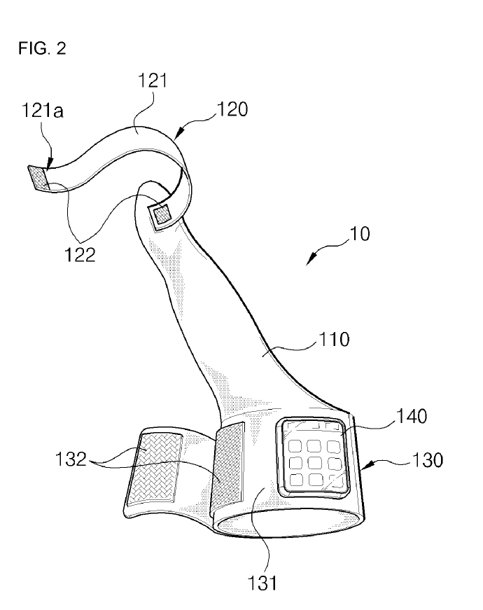

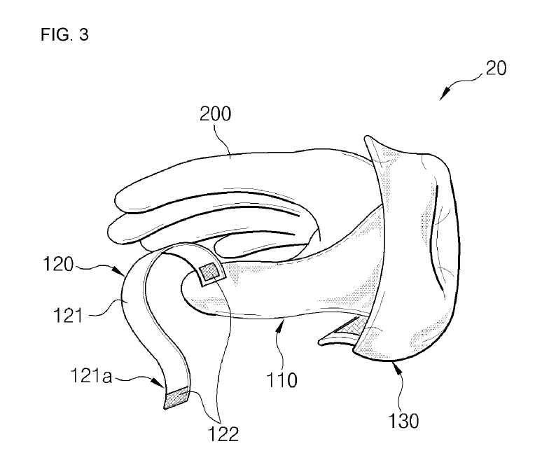

FIG. 2 is a perspective view of a functional glove for holding an ultrasonic probe according to an embodiment of the present invention, and FIG. 3 is a perspective view of a wearing method of the functional glove for holding an ultrasonic probe illustrated in FIG. 2.

As illustrated, a functional glove 10 for holding an ultrasonic probe according to an embodiment of the present invention (hereinafter, referred to as a “functional glove”) includes a body 110, a probe fixing part 120, and a wrist protection part 130. Here, the above-described disposable sanitary gloves 200 may be separately used according to a need of an inspector and is not fixedly coupled to the inside of the body 110. Meanwhile, the functional glove 10 may be used for a disposable purpose, such as the sanitary gloves 200, but may be used a plurality of times separately from the disposable sanitary gloves 200.

The body 110 encompasses at least a part of the thumb and the back of the hand of the inspector who operates the ultrasonic probe P. The body 110 is naturally connected to the wrist protection part 130, which will be described below.

The probe fixing part 120 is coupled to the body 110 so as to encompass and fix the inspector's fingers and the ultrasonic probe P. To this end, the probe fixing part 120 may include a fixing band 121 and an adhesive unit 122.

The fixing band 121 encompasses and fixes the inspector's fingers and the ultrasonic probe P when one end 121a of the fixing band 121 is fixed to the body 110. The fixing band 121. Although one end 121a of the fixing band 121 can be fixed to the part of the body 110 corresponding to the inspector's thumb, the position thereof is not limited. The fixing band 121 may have elasticity so as to elastically fix the inspector's fingers and the probe P. Also, the adhesive unit 122 is installed at both ends of the fixing band 12 so as to adhere or detach both ends of the fixing band 121 to or from each other. The adhesive unit 122 may be a Velcro or snap button with which both ends of the fixing band 121 can be detachably coupled to each other. However, embodiments of the present invention are not limited thereto.

The wrist protection part 130 is connected to the body 120 so as to encompass and protect the inspector's wrist. To this end, the wrist protection part 130 may include a protection band 131 and a detachment unit 132.

One end of the protection band 131 is connected to the body 110 so that the protection band 131 encompasses and protects the inspector's wrist. The protection band 131 may extend from the body 110 as one body with the body 110. However, this is just an example, and the protection band 131 may be connected to the body 110 by adhesion or sewing. The protection band 131 may have elasticity so as to elastically press the inspector's wrist. Also, the detachment unit 132 is installed at both ends of the protection band 131 so as to adhere or detach both ends of the protection band 132 to or from each other. The detachment unit 132 may be a Velcro or snap button with which both ends of the protection band 132 can be detachably coupled to each other, but embodiments of the present invention are not limited thereto.

As described above, in the embodiment of the present invention, while the ultrasonic probe P is hold, the inspector's wrist is pressed and fixed by the wrist protection part 130 such that a wrist injury caused by a long diagnosis time can be prevented.

Meanwhile, the functional glove 10 may further include a remote controller 140 that is installed on the wrist protection part 130 and is capable of remotely operating a main body (not shown) of the ultrasonic imaging diagnosis device through short wireless communication. The remote controller 140 may include an operating button or touch screen having a plurality of menus therein. In this case, the inspector may remotely operate the main body using the operating button or the touch screen. That is, while the inspector performs ultrasonic diagnosis by holding the ultrasonic probe P, it is possible to operate the main body without the need of approaching the main body of the ultrasonic imaging diagnosis device such that the inspector can diagnose the subject more conveniently.

FIG. 3 is a perspective view for explaining a wearing method of the functional glove 10 according to an embodiment of the present invention. As illustrated, the disposable sanitary gloves 200 may be separately used so as to prevent infection, which can occur between the inspector and the subject, as demand. However, in the present embodiment, the sanitary gloves 200 are not fixedly coupled to the inside of the body 110.

First, the inspector wears the disposable sanitary gloves 200. Subsequently, the inspector wears the functional glove 10 according to an embodiment of the present invention. To this end, the inspector inserts his/her thumb into the corresponding part of the body 110. Subsequently, after encompassing the protection band 131 of the wrist protection part 130 on the inspectors' wrist part, the inspector adjusts strength exerted by the protection band 132 in the wrist using the adhesive unit 132 such as Velcro. Next, the inspector's fingers and the ultrasonic probe P are fixed using the probe fixing part 120, which will be described later.

Hereinafter, a functional glove for holding an ultrasonic probe according to another embodiment of the present invention will be described.

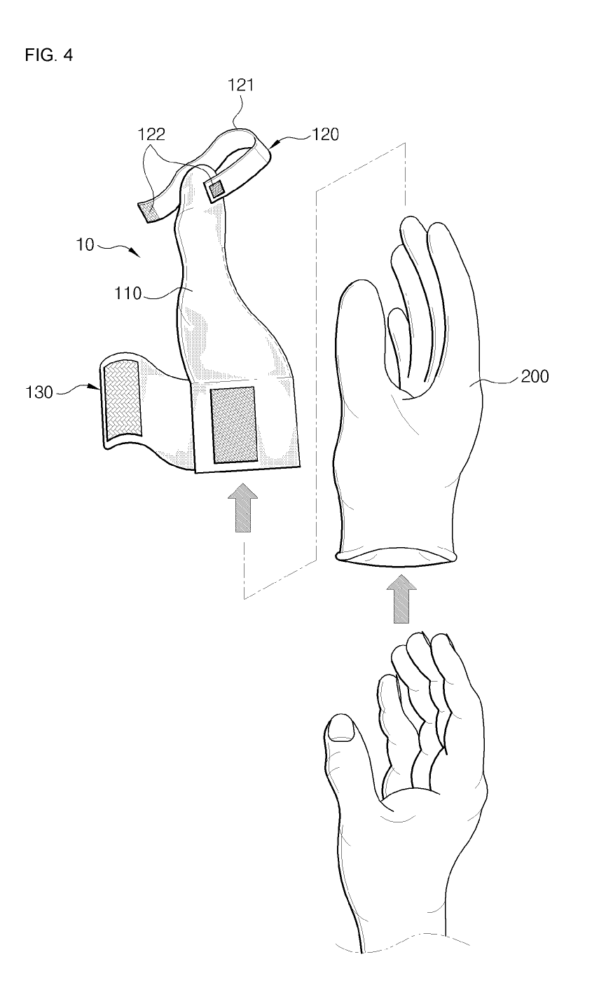

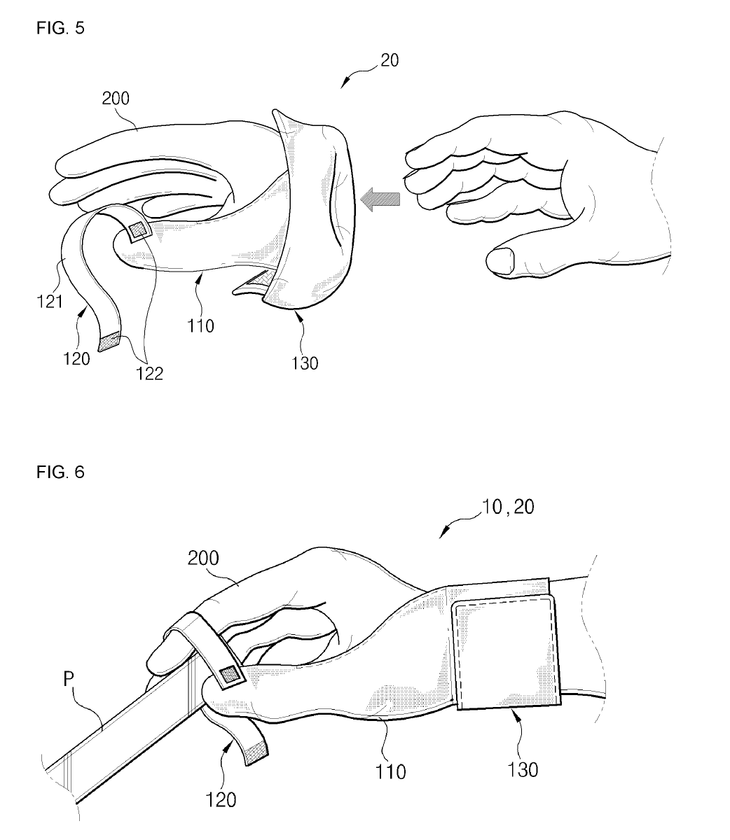

FIG. 4 is a perspective view of a functional glove for holding an ultrasonic probe according to another embodiment of the present invention, and FIG. 5 is a perspective view of a wearing method of the functional glove for holding an ultrasonic probe illustrated in FIG. 4. Here, the same reference numerals as those of FIGS. 2 and 3 represent like elements having the same configurations and effects and thus, a redundant description thereof will be omitted.

Referring to the drawings, a functional glove 20 for holding an ultrasonic probe according to another embodiment of the present invention (hereinafter, referred to as a “functional glove”) includes a body 110, a probe fixing part 120, and a wrist protection part 130. Here, the functional glove 20 further includes sanitary gloves 200 fixedly coupled to the body 110. The sanitary gloves 200 encompass the inspector's fingers and wrist. The sanitary gloves 200 are disposed inside the body 110 and may be fixedly coupled to the inside of the body 110 through an adhesive or sewing.

The functional glove 20 described above may be used for a disposable purpose so as to prevent infection. Also, for wearing convenience of the inspector, the wrist protection part 130 and neck parts of the sanitary gloves 200 may be turned inside out in a direction of the inspector's fingers.

As illustrated in FIG. 5, the inspector may easily wear the functional glove 20 by inserting his/her fingers into the inside of the functional glove 20 of which the wrist protection part 130 and the neck parts of the sanitary gloves 200 are turned inside out. Subsequently, after encompassing the protection band 131 of the wrist protection part 130 in the inspector's wrist part, the inspector adjusts strength exerted by the protection band 131 in the wrist using the adhesive unit 132.

Hereinafter, a method of fixing the ultrasonic probe P to the inspector's hand using the probe fixing part 120 of the functional glove 10 or 20 according to the embodiment of the present invention will be described in detail.

The probe fixing part 120 encompasses the inspector's hand differently according to a method of the inspector holding the ultrasonic probe P. FIGS. 6 and 7 are views for illustrating using states of each of methods of holding an ultrasonic probe in a state in which the functional glove for holding an ultrasonic probe illustrated in FIG. 1 is worn.

First, as illustrated in FIG. 6, when the inspector supports a bottom end of the probe using the inspector's ring finger and little finger by holding sides of the probe with the inspector's thumb, index finger and middle finger, the fixing band 121 encompasses and fixes the probe P with his/her thumb, index finger and middle finger.

Next, as illustrated in FIG. 7, when the inspector holds the ultrasonic probe P so as to encompass the sides of the ultrasonic probe P with his/her palm and five fingers like clenching his/her fist, the fixing band 121 fixes the ultrasonic probe P so as to encompass all of the inspector's five fingers.

As described above, the probe fixing part 120 fixes the inspector's fingers and the probe P while the inspector holds the ultrasonic probe P such that the inspector can hold the probe P without exerting excessive strength in his/her fingers and thus work fatigue of the inspector can be reduced.

In addition, the functional glove for holding the ultrasonic probe according to the present invention can also be applied to an existing ultrasonic probe and can be worn regardless of the inspector's holding method so that high utilization of various ultrasonic diagnosis devices can be achieved.

While the invention has been shown and described with reference to certain example embodiments thereof, it will be understood by those skilled in the art that various changes in form and details may be made therein without departing from the spirit and scope of the invention as defined by the appended claims.

1. A functional glove for holding an ultrasonic probe, comprising:
a body for encompassing the thumb and at least a part of the back of the hand of an inspector operating the ultrasonic probe;
a probe fixing part coupled to the body so as to encompass and fix the fingers of the inspector and the ultrasonic probe; and
a wrist protection part connected to the body so as to encompass and protect the wrist of the inspector.

2. The functional glove for holding an ultrasonic probe of claim 1, further comprising a remote controller that is installed on the wrist protection part and is capable of remotely operating a main body of an ultrasonic imaging diagnosis device through short wireless communication.

3. The functional glove for holding an ultrasonic probe of claim 1, further comprising sanitary gloves disposed inside the body so as to encompass the fingers and the back of the hand of the inspector and fixedly coupled to the body.

4. The functional glove for holding an ultrasonic probe of claim 3, wherein the wrist protection part and neck parts of the sanitary gloves are turned inside out in a direction of the fingers of the inspector.

5. The functional glove for holding an ultrasonic probe of claim 1, wherein the probe fixing part comprises:
a fixing band having one end fixed to the body so as to encompass and fix the fingers of the inspector and the ultrasonic probe; and
an adhesive unit installed at both ends of the fixing band and configured to adhere or detach both ends of the fixing band to or from each other.

6. The functional glove for holding an ultrasonic probe of claim 5, wherein the fixing band has one end fixed to a part of the body corresponding to the thumb of the inspector and has elasticity.

7. The functional glove for holding an ultrasonic probe of claim 5, wherein the adhesive unit comprises a Velcro or snap button with which both ends of the fixing band are detachably coupled to each other.

8. The functional glove for holding an ultrasonic probe of claim 1, wherein the wrist protection part comprises:
a protection band having one end connected to the body so as to encompass and protect the wrist of the inspector; and
a detachment unit installed at both ends of the protection band and configured to adhere or detach both ends of the protection band to or from each other.

9. The functional glove for holding an ultrasonic probe of claim 8, wherein the protection band extends from the body as one body with the body and has elasticity.

10. The functional glove for holding an ultrasonic probe of claim 8, wherein the detachment unit comprises a Velcro or snap button with which both ends of the protection band are detachably coupled to each other.
